SEQUENCE LISTING (2 sequences)

SEQ ID NO: 1 - DNA, 27 bp, Artificial Sequence
  Description of Artificial Sequence Synthetic primer
tataagaaactactaagcacccaaagg

SEQ ID NO: 2 - DNA, 27 bp, Artificial Sequence
  Description of Artificial Sequence Synthetic primer
aagaaaggagtctaagtgactcaacag

(19) United States
(12) Patent Application Publication
(10) Pub. No.: US 2006/0286557 A1
(43) Pub. Date: Dec. 21, 2006

(54) Combined lysis and PCR buffer

(76) Inventors: Lee Scott Basehore, Lakeside, CA (US); Natalia Novoradovskaya, San Diego, CA (US); Jeffery Carl Braman, Carlsbad, CA (US)

Correspondence Address: LATIMER IP LAW, LLP, 13873 PARK CENTER ROAD, SUITE 122, HERNDON, VA, 20171, US

(21) Appl. No.: 11/152,773

(22) Filed: Jun. 15, 2005

(51) Int. Cl. C12Q 1/68; C12P 19/34

(52) U.S. Cl. 435/6; 435/91.2; 435/270

(57) ABSTRACT

The present invention provides compositions, methods, and kits for lysing cells, storing nucleic acids, amplifying nucleic, and analyzing nucleic acids. Among other things, the compositions, methods, and kits are suitable for one-step lysis and amplification of nucleic acid sequences of interest. In general, the compositions comprise TCEP and a non-ionic detergent, such as Triton X-100.

### BACKGROUND OF THE INVENTION

1. Field of the Invention

The present invention relates to the field of molecular biology. More specifically, the present invention relates to compositions and methods for lysing cells, preparing nucleic acids for amplification or other manipulations, amplifying nucleic acids, and storing nucleic acids.

2. Description of Related Art

Compositions for lysis of cells, including both prokaryotic and eukaryotic cells, are known in the art. Typically, the compositions include organic solvents or detergents that dissolve or disrupt cellular membranes or walls (when chemical lysis is used) or that enable cellular components of interest to be isolated from other components (when either chemical or mechanical lysis is used). Among the mechanical methods known for lysing cells, mechanical disruption (e.g., mechanical blender, glass beads, grinding of frozen cells), liquid homogenization (e.g., French pressure cell, Dounce homogenizer), high frequency sound waves (e.g., sonication), and freeze/thaw cycles (e.g., dry ice baths, liquid nitrogen baths) are the most commonly used. Common chemical methods, which are used alone or in combination with mechanical methods, include use of hypotonic buffers and use of cell wall degrading enzymes, such as lysozyme. Depending on the ultimate use for the lysed cells, other macromolecule-degrading substances are often included in the lysis buffers. For example, if cells are being lysed for capture of nucleic acids, proteases can be added to degrade proteins that might interfere with isolation of the nucleic acids. Conversely, if proteins are of interest, DNases or RNases may be added to remove the contaminating nucleic acids, while protease inhibitors can be added.

Protocols for lysis of cells and isolation of nucleic acids are known in the art. Many involve exposing the cells to organic solvents, such as phenol and chloroform, and later removing traces of these organics to enable enzymatic manipulation of the nucleic acids. Others involve exposing cells to detergents and other substances that dissolve membranes and/or cause osmotic pressure to lyse cells. Among the many detergents used is sodium dodecyl sulfate (SDS). The combination of SDS and sodium hydroxide has been known for years to be suitable for lysis of prokaryotic cells. Purification of nucleic acids, particularly DNA, follows by removal of cellular debris and the lysis agents.

For example, extraction of RNA is often done using a guanidine-isothiocyanate phenol:chloroform extraction method in which protein and DNA are partitioned into the organic layer, while RNA is partitioned into the aqueous layer. A subsequent precipitation of the RNA with an alcohol, such as isopropanol, results in purified RNA. In some variations of this protocol, β-mercaptoethanol (BME) is included in the lysis buffer. Cell lysis and DNA isolation also often relies on phenol:chloroform extraction. However, some methods avoid the use of organics, and rely on preferential precipitation of proteins by a salt, such as with sodium chloride, followed by precipitation of the nucleic acid with alcohol. Other methods rely on solid resins, such as silica-based resins, to bind proteins and separate them from nucleic acids, particularly DNA.

Cells are commonly lysed with N-lauryl sarcosyl detergent and guanidine thiocyanate or lithium chloride (both chaotrophic salts). Nucleic acids are then isolated out of this lysate solution by passing the lysate over a glass fiber filter or mixing it with charged polymeric beads, either of which will bind the nucleic acids. Subsequent washing and elution of the nucleic acids is required.

Where amplification of nucleic acids liberated from the cells is desired, the lysis compositions typically contain substances that inhibit nucleic acid degrading enzymes, such as DNases and RNases, or the composition comprising the nucleic acids are treated with heat to inactivate the nucleic acid degrading enzymes. Among the most common of such substances are diethyl pyrocarbonate (DEPC), phenol, proteases, ionic detergents, and antibody cocktails. Heating to a temperature sufficient to inactivate or denature proteins is a viable step for minimizing nucleic acid degradation. However, this step is often problematic where ribonucleic acids are of interest.

TCEP (Tris(2-carboxyethyl)phosphine) is a sulfhydryl reductant that has found use in stabilization of proteins, such as those to be studied for protein biochemistry or for use in crystallization. For example, a recent publication by Vasiljev et al. discloses the use of buffers containing TCEP for solubilization of mitochondria, and subsequent purification of mitochondrial proteins. (Vasiljev, A. et al., Molecular Biology of the Cell 15:1445-1448, March 2004.) However, this reference does not approach the use of such buffers in lysing cells, or its usefulness in reactions for amplification of nucleic acids.

In addition, TCEP is known in the art as a useful component of compositions for solubilization of proteins, including solubilization for later crystallization. For example, the Proteomics Core Facility at the University of Cincinnati (http://www.med.uc.edu/proteomics/solubilization.cfm) discloses a buffer called “ASB-14” for solubilization and rehydration of proteins. The buffer contains among other things TCEP and Triton X-100 as components. This buffer, however, is not disclosed or suggested as having usefulness for any purpose other than solubilization and rehydration of proteins. In view of the fact that the ASB-14 buffer contains 7 molar (M) urea, it is unlikely to have such usefulness. Furthermore, buffers for purification of proteins for later crystallization are known. One such buffer is disclosed at http://www.sgc.ox.ac.uk/structures/MM/HSD11B1_{—}2be1_MM.html. This buffer contains, among other things, TCEP and Triton X-100. The buffer is disclosed as useful for solubilization and purification of proteins. However, it is not disclosed as suitable for any further purposes, including amplification of nucleic acids.

TCEP has also been proposed by Rhee and Burke as a replacement for dithiothreitol (DTT) in protocols involving nucleic acids (Rhee, S. S., and D. H. Burke, Anal. Biochem. 325:137-143, 2004). These investigators determined that TCEP was more stable than DTT at neutral to basic pH and at elevated temperatures. They also determined that TCEP could stabilize RNA at high temperatures and neutral pH to a greater extent than DTT. In view of these findings, they concluded that TCEP, rather than DTT, could be used as a sulfhydryl reductant in nucleic acid and thiophosphate chemistry. However, these investigators did not report any research on the characteristics of TCEP at an acidic pH, nor its compatibility with enzymes or other substances typically used in lysis of cells or in molecular biology and protein biochemistry assays and protocols.

Burns et al. (Burns, J. A., et al., J. Org. Chem. 56:2648-2650, 1991) reported the synthesis and characterization of TCEP. They conclude that TCEP reduces disulfides rapidly and completely in water at pH 4.5. They also report that TCEP is a selective reducing agent for representative dialkyl disulfides in aqueous solutions. However, Burns et al. does not approach the usefulness of TCEP in lysing cells or stabilizing nucleic acids, or its compatibility with enzyme reactions.

Non-ionic detergents contain uncharged, hydrophilic head groups. They have found wide use in molecular biology, cellular biology, and protein biochemistry, particularly in situations requiring the breaking of lipid-lipid bonds, such as when solubilizing membranes and membrane-bound proteins. They are often the detergent of choice where ionic interactions between the detergent and one or more component in a mixture is undesirable, or where interaction between the detergent and a purification reagent (e.g., a matrix for column chromatography) is undesirable. Detergents have been used in many different concentrations for purification of different proteins, and for solubilization of different membranes. There presence in compositions is often problematic after their primary use has been completed, and they are often removed prior to enzymatic reactions to minimize any deleterious effects they might have on the reactions.

Various buffers and protocols are available to lyse cells and produce nucleic acids that are suitable for analysis. For example, Ambion (www.ambion.com) sells a product called Cells-to-cDNA™, which is reported to produce cDNA from mammalian cells in culture without an RNA isolation step. In the protocol provided by Ambion, crude cell lysate is produced by exposing cells to a cell lysis buffer at a high temperature. Heating is used to inactivate endogenous RNases. After heating, DNA is degraded with DNase I, and the mixture heated again to inactivate the DNase I.

Ambion also sells a product called Cells-to-Signal™, which is based on the Cells-to-cDNA™ technology, but which eliminates the required heating steps that are part of the Cells-to-cDNA™ protocol. The Cells-to-Signal™ technology uses a proprietary buffer that is reported to permit lysis of cells and analysis of released nucleic acids without the need for isolation of the nucleic acid of interest. Ambion does not disclose the components of the buffer used in the Cells-to-Signal™ product, but it appears to comprise a non-reducing, low pH detergent buffer.

In addition, Microzone (www.microzone.co.uk) sells a product called MicroLYSIS®. The MicroLYSIS® product is reported to provide a single cell lysis buffer that is compatible with PCR. According to the MicroLYSIS® protocol, cells are lysed and nucleic acids obtained by repeated heating and cooling cycles in the buffer.

Although numerous compositions for lysing cells are known in the art, and some of those compositions are disclosed as having use in performing PCR, a need still exists in the art for compositions that provide not only cell lysis, but permit high quality amplification of nucleic acids from the lysed cells.

### SUMMARY OF THE INVENTION

The present invention addresses needs in the art by providing compositions that are suitable for lysis of cells and analysis of nucleic acids. Thus, the present invention provides compositions that are suitable for lysis of cells and amplifying nucleic acids liberated from those cells, subcloning of liberated nucleic acids, copying of liberated nucleic acids, ligating liberated nucleic acids, sequencing liberated nucleic acids, labeling liberated nucleic acids, and the like. The compositions further can permit long-term storage of cell lysates while maintaining the nucleic acids in a state that permits robust usage, such as high-quality amplification, even after such long-term storage. Methods and kits based on the compositions are likewise provided, including those for rapid lysis of cells and amplification of nucleic acids contained in the cells.

In a first aspect, the invention provides a composition, also referred to herein at times as a buffer, that is suitable for lysis of cells, storage of nucleic acids, amplification of nucleic acids, and other processes commonly performed on nucleic acids for various molecular biology purposes. In its broadest form, the buffer comprises tris(2-carboxyethyl)phosphine (TCEP) and a non-ionic detergent. Various embodiments of the compositions comprise other substances that are compatible or useful in lysing cells, storing nucleic acids, amplifying nucleic acids, purifying nucleic acids, and/or other procedures for analysis of nucleic acids. Although not so limited, the compositions of the invention can be considered one-step reagents for preparation, storage, amplification, and/or purification of nucleic acids.

In a second aspect, the invention provides a method of lysing at least one cell using a composition of the invention. The method of lysing generally comprises contacting at least one cell with a composition of the invention for a sufficient amount of time to cause the cell to lyse. In various embodiments, additional optional steps are included in the method of lysing cells, such as storing the cell lysate for a period of time prior to use of the lysate. Likewise, other exemplary additional steps can include amplifying one or more nucleic acids in the cell lysate, and/or purifying the nucleic acid(s).

In a third aspect, the invention provides a method of amplifying one or more nucleic acids. In general, the method comprises exposing at least one nucleic acid to a composition of the invention, and amplifying the nucleic acid. In various embodiments, amplifying is by a PCR method, such as QPCR or RTPCR. In certain embodiments, one or more control reactions are included, such as a control reaction to permit normalization of the amount of nucleic acid being amplified with respect to other amplification reactions that are being performed concurrently, or with respect to a standard amplification curve. In embodiments, the method of amplifying can comprise lysing cells containing the nucleic acid(s) prior to amplification. It has surprisingly been found that a single buffer of the invention can be suitable for both lysis of cells and amplification of nucleic acids.

In a fourth aspect, the invention provides a method of stabilizing at least one nucleic acid. In general, the method of stabilizing comprises exposing at least one nucleic acid to a composition of the invention. It has surprisingly been found that the compositions of the invention can be used to store nucleic acids for relatively long periods of time without significant loss of nucleic acid or nucleic acid quality. Thus, nucleic acids can be stored for relatively long periods of time and subsequently used for analysis, such as by a PCR technique. In embodiments, the method of stabilizing a nucleic acid includes exposing a cell containing the nucleic acid to a composition of the invention for a sufficient amount of time for lysis of the cell to occur, then maintaining the cell lysate, or a fraction of the cell lysate containing nucleic acids, for a period of time prior to use of the nucleic acids. In certain embodiments, the stabilized nucleic acid is subsequently used for amplification, such as by a PCR technique.

In a fifth aspect, the invention provides kits. In general, the kits contain a composition of the invention. The kits can further comprise one or more substances, materials, reagents, etc. that can be used for lysis of cells, storage of nucleic acids or cell lysates, amplification of nucleic acids, or purification or quantitation of nucleic acids. In embodiments, some or all of the materials, reagents, etc. necessary to lyse cells, amplify nucleic acids, and/or purify and/or quantitate nucleic acids are included in the kit.

### BRIEF DESCRIPTION OF THE DRAWINGS

The accompanying drawings, which are incorporated in and constitute a part of this specification, illustrate exemplary embodiments of the invention, and together with the written description, serve to explain certain principles of the invention. The drawings are not to be considered as limiting the scope of the invention in any way.

FIG. 1 depicts results of an agarose gel electrophoresis of amplification products obtained using a currently available commercial cell lysis and amplification buffer, a buffer comprising salicylic acid and Triton X-100, a buffer according to the present invention, phosphate buffered saline (PBS), and isolated Jurkat RNA. NTC stands for no template control (negative control).

FIG. 2 depicts amplification plots of QRT-PCR reactions on HeLa lysates stored at 4° C., −20° C., and −80° C. for approximately 72 hours.

FIG. 3 depicts amplification plots of QRT-PCR reactions on HeLa lysates stored at 4° C., −20° C., and −80° C. for 17 days.

FIG. 4 depicts an electropherogram of capillary electrophoresis analysis of RNA obtained from lysis of HeLa cells in a buffer of the invention.

FIG. 5 depicts an electropherogram of capillary electrophoresis analysis of RNA obtained from lysis of HeLa cells in a buffer of the invention, after storage at −20° C. for 3 weeks.

FIG. 6 depicts amplification plots of QRT-PCR of Jurkat lysates that have been stored at room temperature from 0 to 168 hours.

FIG. 7 depicts an amplification plot standard curve for an internal control primer set according to the invention.

FIG. 8 depicts a standard curve developed from the amplification plot of FIG. 7.

FIG. 9A depicts amplification plots of different amounts of cell lysates, representing from 4.8 cells to 600 cells, in a buffer of the invention and using a primer set that specifically amplifies unique sequences on human genomic nucleic acid.

FIG. 9B depicts amplification plots of different amounts of cell lysates, representing from 8 cells to 1,000 cells, in a buffer of the invention and using the same primer set as in FIG. 9A.

FIG. 10 depicts amplification plots of QRT-PCR reactions of cheek cell lysates obtained from swabs of human cheek (mouth mucosal) cells.

FIG. 11 depicts amplification plots of QRT-PCR reactions of whole blood lysates.

FIG. 12 depicts amplification plots of QRT-PCR reactions of mouse liver homogenates.

FIG. 13 depicts amplification plots of QRT-PCR reactions of HeLa cell lysates and purified mRNA from HeLa cell lysates.

### DETAILED DESCRIPTION OF VARIOUS EMBODIMENTS OF THE INVENTION

Reference will now be made in detail to various exemplary embodiments of the invention. These exemplary embodiments are provided to better explain certain details that may apply to some aspects of the invention, but are not to be considered as limiting the scope of the invention to any particular configuration of composition components or method steps.

In a first aspect, the invention provides a composition. In its most basic form, the composition comprises tris(2-carboxyethyl)phosphine (TCEP) and at least one non-ionic detergent. Thus, in one embodiment, it consists of TCEP and at a non-ionic detergent. It has been surprisingly found that the combination of these two substances provides a composition that can be used for numerous purposes, including lysis of cells, stabilization of nucleic acids, storage of nucleic acids, purification of nucleic acids, or a combination of two or more of these, without significant degradation of the nucleic acids. It has also been surprisingly found that the composition is suitable for amplification of nucleic acids by PCR methods, such as reverse transcriptase PCR (RTPCR) and quantitative PCR (QPCR), even after long-term storage of the target nucleic acid in a composition comprising the TCEP and non-ionic detergent. To the inventors' knowledge, this is the first time that a single composition comprising these two components has been recognized as capable of use for lysis of cells, amplification of nucleic acids, or long term stable storage of nucleic acids. Furthermore, to the inventors' knowledge, this is the first time that a single composition comprising these two components has been recognized as capable of providing a combination of two or more of these characteristics.

TCEP can be included in a composition of the invention at any suitable concentration. For example, TCEP can be present in a concentration from 0.1 mM to 100 mM. It thus can be present in a concentration from 0.5 mM to 50 mM, such as from about 1 mM to about 10 mM. For example, it can be present in a concentration of 1 mM, 2 mM, 3 mM, 4 mM, 5 mM, 6 mM, 7 mM, 8 mM, 9 mM, 10 mM, or any fraction thereof. Because compositions of the invention can be provided in a “working” concentration or as a concentrated “stock solution” that is diluted prior to use or diluted significantly upon addition to other components in the composition of the invention, the above concentrations are not limited to final concentrations of a reaction mixture (e.g., lysis reaction), but can represent compositions packaged or prepared with the understanding that they might be altered before use.

It is to be noted at this point that each value stated in this disclosure is not, unless otherwise stated, meant to be precisely limited to that particular value. Rather, it is meant to indicate the stated value and any statistically insignificant values surrounding it. As a general rule, unless otherwise noted or evident from the context of the disclosure, each value includes an inherent range of 5% above and below the stated value. At times, this concept is captured by use of the term “about”. However, the absence of the term “about” in reference to a number does not indicate that the value is meant to mean “precisely” or “exactly”. Rather, it is only when the terms “precisely” or “exactly” (or another term clearly indicating precision) are used is one to understand that a value is so limited. In such cases, the stated value will be defined by the normal rules of rounding based on significant digits recited. Thus, for example, recitation of the value “10” means any whole or fractional value between 9.5 and 10.5, whereas recitation of the value “exactly 100” means 99.5 to 100.4.

The compositions of the invention comprise at least one non-ionic detergent. Accordingly, they can comprise one or more of any non-ionic detergent. Examples of suitable non-ionic detergents include, but are not limited to: BIGCHAP (N,N-bis-(3-D-Gluconamidopropyl)cholamide) or deoxy-BIGCHAP (N,N-bis(3-Gluconamidopropyl)deoxycholamide); Decanoyl-N-methylglucamide; n-Decyl α-D-Glucopyranoside; n-Decyl β-D-Glucopyranoside; n-Decyl β-D-Maltopyranoside; Digitonin; n-Dodecyl β-D-Glucopyranoside; n-Dodecyl α-D-Maltoside; n-Dodecyl β-D-Maltoside; heptanoyl-N-methylglucamide; n-Heptyl β-D-Glucopyranoside; N-Heptyl β-D-Thioglucopyranoside; n-Hexyl β-D-Glucopyranoside; 1-Monooleoyl-rac-glycerol; Nonanoyl-N-methylglucamide; n-Nonyl α-D-Glucopyranoside; n-Nonyl β-D-Glucopyranoside; Octanoyl-N-methylglucamide; n-Octyl α-D-glucopyranoside; n-Octyl β-D-Glucopyranoside; Octyl β-D-Thiogalactopyranoside; Octyl β-D-Thioglucopyranoside; Polyoxyethylene Esters (such as 8-stearate polyoxyethylene ester (Myrj 45), 40-stearate polyoxyethylene ester (Myrj 52), 50-stearate polyoxyethylene ester (Myrj 53), and 100-stearate polyoxyethylene ester (Myrj 59)), Polyoxyethylene Ethers (such as those containing one or more ethyl groups, methyl groups, pentyl groups, cetyl groups, stearyl groups, oleyl groups, hexyl groups, octyl groups, decyl groups, lauryl groups, myristyl groups, heptyl groups, tridecyl groups, isohexadecyl groups, and combinations thereof); Polyoxyethylenesorbitan esters (such as those containing one or more monolaurate groups, monooleate groups, monopalmitate groups, monostearate groups, trioleate groups, and tristearate groups, and combinations thereof, including, but not limited to the “Tween” series of detergents); Sorbitan esters (such as those containing one or more monolaurate groups, monooleate groups, monopalmitate groups, monostearate groups, sesquioleate groups, trioleate groups, tristearate groups, and combinations thereof); Terigol; n-Tetradecyl β-D-Maltoside; the Triton series of detergents, including, but not necessarily limited to, Triton X-100 (t-Octylphenoxypolyethoxyethanol) and its derivatives, Triton X-114, Triton X-405, Triton X-101, Triton N-42, Triton N-57, Triton N-60, Triton X-15, Triton X-35, Triton X-45, Triton X-102, Triton X-155, Triton X-165, Triton X-207, Triton X-305, Triton X-705-70 and Triton B-1956; Nonylphenyl Polyethylene Glycol (Nonidet P-40; NP-40, Igepal CA630); Tyloxapol; n-Undecyl β-D-Glucopyranoside, and any non-ionic Octylphenol Ethoxylate surfactant. Combinations of two or more of these or other non-ionic detergents/surfactants are encompassed within the term “non-ionic detergent”.

Exemplary compositions of the invention comprise TCEP and a non-ionic detergent, such as Triton X-100, in the following amounts, respectively: 1 mM and 0.5%; 1 mM and 1%; 1 mM and 2.5%; 1 mM and 5%; 2.5 mM and 0.5%; 2.5 mM and 1%; 2.5 mM and 2.5%; 2.5 mM and 5%; 5 mM and 0.5%; 5 mM and 1%; 5 mM and 2.5%; 5 mM and 5%; 7.5 mM and 0.5%; 7.5 mM and 1%; 7.5 mM and 2.5%; 7.5 mM and 5%; 10 mM and 0.5%; 10 mM and 1%; 10 mM and 2.5%; 10 mM and 5%. In certain embodiments, the compositions comprise 5 mM TCEP and 1% Triton X-100. Multiples of these combinations of amounts are also envisioned as exemplary combinations of concentrations. For example, concentrated stock solutions can be formulated as a 20× stock, 10× stock, 5× stock, or 2× stock. Thus, exemplary compositions can comprise 100 mM TCEP and 20% non-ionic detergent(s), 50 mM TCEP and 10% non-ionic detergent(s), 25 mM TCEP and 5% non-ionic detergent(s), and 10 mM TCEP and 2% non-ionic detergent(s).

Although the composition may comprise one or more other substances, and those substances are not limited by the exemplary substances disclosed herein, the composition will typically contain a solvent, such as water, an organic solvent, or both. Although it is preferred that the solvent used be as pure as possible or practicable, solvents of any purity may be used. Thus, where water is included in the composition, it may be distilled water, double-distilled water, de-ionized water, sterilized water, or any combination thereof. The solvent, be it water or any other solvent or combination of water and any other solvent, may be treated before use to reduce or eliminate one or more chemical or biochemical activities, such as, but not limited to nuclease (e.g., RNase, DNase) activities. Likewise, the composition may be treated with sterilization techniques or with chemicals or biologicals, etc. to sterilize the composition or to reduce or eliminate one or more undesirable chemical or biochemical activities (e.g., RNase, DNase, etc.).

As noted above, the composition can comprise other substances in addition to TCEP and a non-ionic detergent. For example, it can comprise one or more salts, such as a sodium salt, a potassium salt, a magnesium salt, a manganese salt, a zinc salt, a cobalt salt, or a combination of two or more of these salts. Specific exemplary salts include sodium chloride, magnesium chloride, manganese chloride, and potassium chloride. The salts may be added in any suitable amount and for any reason, including, but not limited to, as an aid in lysis of cells, for moderation of detergent cloud point and foam level, and for improved function of reagents involved in amplification of nucleic acids.

The compositions of the invention are suitable for lysis of cells. Thus, in embodiments, compositions of the invention comprise cells. That is, the composition may be a buffer or stock solution. The buffer may be added to one or more cells to create another composition of the invention. In compositions comprising cells, the TCEP may be present in a concentration of from about 0.05 mM to about 100 mM. In some embodiments, for lysis of cells, the buffer is added at or near a 1× concentration, owing to the fact that the volume of cells is relatively small compared to the amount of buffer added. For example, in many embodiments, from 1 to 50,000 cells are treated with the buffer in a single vessel, using 100 microliters (ul) of buffer. Because the volume of cells is relatively small compared to the volume of buffer added, the resulting composition comprises approximately the same concentration of TCEP, non-ionic detergent, and other components (if present) as the original buffer. Accordingly, for compositions comprising cells or cell lysates, the concentrations of TCEP, non-ionic detergent, and other optional components are those described above with regard to the base composition of the invention.

The number of cells included in the composition (before lysis), and thus the volume of cells, can vary depending on cell type. Numerous cell types from various organisms, which can be isolated directly from tissues, grown in culture media, grown in culture media for one to many passages, or from cell lines and tissue cultures, can be included in the compositions of the inventions. Thus, the cells can be samples of, or originate from, tissue samples, including, but not limited to liver tissue, kidney tissue, brain tissue, blood, lymph tissue, and bone tissue. Typically, the number of cells present in a composition ranges from about 1 cell to about 100,000 cells. For example, in embodiments, 50,000 cells are present with a buffer of the invention. In other embodiments, 40,000 cells are present with the buffer, while in yet other embodiments, 30,000 cells, 25,000 cells, 20,000 cells, 15,000 cells, 10,000 cells, 5,000 cells, 2,500 cells, 1,000 cells, 500 cells, 250 cells, 100 cells, 50 cells, 25 cells, or 10 cells are present. Likewise, the amount of cell lysate present in a composition typically represents lysate from about 1 cell to about 100,000 cells, such as from about 1 cell to about 50,000 cells. Exemplary numbers of cells serving as a basis for cell lysate are those given above with regard to the number of cells in a composition.

In its most basic form, the composition comprises TCEP and a non-ionic detergent. It has been surprisingly found that compositions comprising TCEP and a non-ionic detergent can be used for lysis of cells, and in particular, eukaryotic cells, such as those grown in cell culture. Non-limiting examples of cells that can be lysed with compositions of the present invention include HeLa, COS 7, CHO, MCF 7, NIH 3T3, Jurkat, Hep G2, 293 cell lines. Thus, in embodiments, the composition of the present invention comprises TCEP, a non-ionic detergent, and at least one cell. Likewise, in embodiments, the composition of the present invention comprises TCEP, a non-ionic detergent, and cell lysate from at least one cell. As is known in the art, cell lysis methods, under varying conditions, can result in complete lysis of all cells present, or lysis of only a portion of the cells present. Thus, in embodiments, the composition of the present invention comprises TCEP, a non-ionic detergent, at least one lysed cell, and at least one intact or un-lysed cell.

The compositions of the invention are suitable for isolation, storage, and analysis of nucleic acids, including all types of DNA and RNA. As discussed in more detail below, in embodiments the methods of the invention contemplate removing non-nucleic acid cell debris and intact cells after exposure of cells to a composition of the invention. Accordingly, in embodiments, the present invention provides a composition comprising TCEP, at least one non-ionic detergent, and at least one nucleic acid. Such compositions may comprise those components as the predominant non-solvent components of the composition, or they may constitute a minority of the non-solvent components of the composition.

The compositions of the invention have surprisingly been found to permit long-term storage of cell lysates without significant degradation of the nucleic acids present in the sample. Thus, the compositions have proved to be suitable for use as storage buffers for nucleic acids. No components are required in addition to the TCEP and non-ionic detergent to provide the long-term storage capabilities. However, in embodiments, other components are included in the compositions. Exemplary components in the storage buffers include, but are not limited to, cryo-preservative agents, such as sugars (monosaccharides, disaccharides, etc.), organic solvents, such as glycerol, ethanol, isopropanol, or other alcohols, and mixtures thereof.

In addition, the compositions of the invention have surprisingly been found to be suitable for amplification of nucleic acids. It has been found that neither the TCEP nor the non-ionic detergent interfere with the specificity or sensitivity of amplification of nucleic acids by PCR techniques. Thus, in embodiments, compositions of the invention comprise at least one reagent that is used in PCR. For example, in embodiments, the compositions comprise at least one primer for amplification of a target nucleic acid. The composition may also comprise betaine, dimethylsulfoxide (DMSO), polyethylene glycol (PEG), and/or tetramethyl ammonium chloride (TMAC). Also, certain embodiments comprise a probe, such as a TaqMan probe, a molecular beacon, or a scorpion probe, in the composition. In embodiments, the compositions comprise at least one polymerase, including, but not limited to a thermostable polymerase and a reverse transcriptase. In embodiments, the compositions comprise one or more nucleotides or nucleotide analogs, which can be incorporated into a growing nucleic acid chain being synthesized by a polymerase. In embodiments, a detecting agent for amplified nucleic acids is present in the composition. Such detecting agents are well known, and include, without limitation, SYBR green, radiolabeled nucleotides, a TaqMan probe, a molecular beacon, a scorpion probe, or any other suitable fluorescent or chemiluminescent compound or combination of compounds. In essence, because the compositions of the invention are suitable for amplification of nucleic acids, any one or more reagent that is known to be useful in an amplification reaction, such as one of the many PCR techniques, can be present in a composition of the invention.

In embodiments, one or more exogenous nucleic acids (i.e., a nucleic acid not originating in whole from a cell that was lysed with the composition of the invention) is included in the composition. The exogenous nucleic acid(s) can be included as an internal control for amplification efficiency, sensitivity, etc.

As discussed above, various embodiments of the compositions comprise other substances that are compatible or useful in lysing cells, amplifying nucleic acids, and/or purifying nucleic acids. It is envisioned that any substance that does not interfere with cell lysis, nucleic acid storage, nucleic acid amplification, or a combination of two or more of these, may be included in the composition of the invention. Thus, the compositions of the invention can be considered one-step reagents for preparation, storage, amplification, and/or purification of nucleic acids.

The pH of the composition may be any suitable pH. However, when the composition is to be used for storage of nucleic acids, it is preferred that the pH be maintained below 7. For example, a pH of 6.0-2.0 has been found to be suitable for long-term storage of DNA and RNA. A pH range of 5.0-2.0 has likewise been found to be suitable, as has a pH range of 4.0-2.5. In certain embodiments, a pH of 2.5 is used. In other embodiments, a pH of 2.0, 2.1, 2.2, 2.3, 2.4, 2.6, 2.7, 2.8, 2.9, or 3.0 is used. In some embodiments, a pH of 7.0 or below is contemplated. In other embodiments, a pH of 7.5 or below is contemplated. In yet other embodiments, a pH of 8.0 or below is contemplated. While not being limited to any particular mode of action, it has been observed that compositions having a pH of 8.0 or below, and in particular, 7.0 or below, 6.5 or below, 6.0 or below, and 5.0 or below provide advantages in stability of nucleic acids, particularly over extended periods of time.

In a second aspect, the invention provides a method of lysing cells using a composition of the invention. The method comprises contacting at least one cell with a composition of the invention for a sufficient amount of time to cause the cell to lyse.

The cell can be any eukaryotic cell. In exemplary embodiments, the cell is a eukaryotic cell other than a yeast cell. The cell can be any cell of interest, including, but not limited to, mammalian cells, avian cells, amphibian cells, reptile cells, and insect cells. For example, the cell can be a human cell, a monkey cell, a rat cell, a mouse cell, a dog cell, a cat cell, a pig cell, a horse cell, a hamster cell, a rabbit cell, a frog cell, or an insect cell.

By the term “at least one cell”, it is meant not only a single cell, but a single cell type. Thus, two or more cells can mean not only two or more cells of the same cell type, but one or more cell of two different cell types. Unless otherwise specifically noted, it is not relevant whether a population of a single cell type is present or a population of two or more cell types is present. Regardless, the methods of the invention (including those discussed below) will provide the stated effects. Furthermore, the term “at least one cell” and “a cell” are, unless otherwise noted, used interchangeably herein to define a single cell, a collection of a single type of cell, or a collection of multiple types of cells, at least one cell of each type being present.

The amount of time the cell is exposed to the composition comprising TCEP and a non-ionic detergent is an amount of time sufficient to cause lysis of at least one cell. Due to the chemical nature of the lysis reaction, it is envisioned that the time can be quite short, on the order of 1 second or less. However, the time is not so limited. Indeed, because it has been found that the compositions of the invention can provide long-term storage of nucleic acids, the time for lysis of cells can be relatively long. Suitable times can range from 1 second or less to minutes, hours, or days. Exemplary times for exposure include, but are not limited to, 10 seconds, 15 seconds, 20 seconds, 25 seconds, 30 seconds 35 seconds, 40 seconds, 45 seconds, 50 seconds, 55 seconds, 1 minute, 90 seconds, 2 minutes, 5 minutes, 10 minutes, 20 minutes, and 30 minutes.

In various embodiments, additional optional steps are included in the method of lysing. While it is possible that lysis will occur in a quiescent sample, to expedite lysis, the cells can be exposed to one or more mechanical disruption techniques. Any known mechanical disruption technique may be used, including, but not limited to, vortexing, repeated pipeting, inversion, shaking, and stirring of the cell-containing composition. Thus, lysis can be accomplished, at least in part, by homogenizing (e.g., with a blender) and bead beating. Depending on the ultimate use of the cell lysate, the mechanical techniques, when used, may be applied gently to minimize shearing stresses on the nucleic acids. Likewise, lysis can be accomplished, at least in part, through the action of biological or biochemical substances. For example, lysis can be accomplished in the presence of a proteinase, such as Proteinase K.

Another optional step in the method of lysing cells is storing the cell lysate for a period of time before use. Because the compositions of the invention are suitable for long-term storage of cell lysates, and particularly nucleic acids, the step of lysing may be continued for a long period of time. As it is often difficult to determine with precision whether each and every cell in a composition has been lysed, and because compositions of the invention can comprise both intact cells and cell lysates, the method of lysis, when it includes a storage step, can overlap with the method of storing cell lysates or nucleic acids (discussed below). Under such situations, it is not critical which method is considered as being used, as long as it is understood that one or both of the methods of the invention are being used. In embodiments where the cell lysate is stored, it can be stored at any number of temperatures. For example, it can be stored at relatively high temperatures (e.g., 37° C.), at room temperature (e.g., 22° C.-25° C.), in the refrigerator (e.g., 4° C.), frozen (e.g., −20° C.), or deep frozen (e.g., −80° C. or lower).

The method of lysing cells can also include one or more steps that result in separation of cell components from other cell components. Thus, for example, the method may comprise centrifuging the cell lysate to remove unlysed cells, cell membranes and proteins from nucleic acids. While not preferred, it can also include precipitation of one or more cellular component from others, for example, though addition of one or more salts, organic solvents (e.g., alcohol), or through heat treatment and subsequent centrifugation. Other techniques for separating cellular components from each other are known to those of skill in the art, and any suitable technique may be used, each being selected based on the desired outcome. Performance of these techniques, and selection of the appropriate technique are well within the skill level of those of skill in the art.

The method of lysing may also include manipulation of one or more cell lysate component. Thus, the method may include purification of one or more protein from the lysate, purification of one or more nucleic acid from the lysate, or amplification of one or more nucleic acid from the lysate. In essence, embodiments of the lysing method include any and all procedures that are known for use with cell lysates.

It has been surprisingly found that compositions comprising TCEP and at least one non-ionic detergent are suitable for numerous different enzymatic reactions involving nucleic acids. Thus, the method of lysing may optionally include one or more steps involved in nucleic acid modification, analysis, or amplification procedures. For example, all nucleic acid polymerases tested have shown to be functional in the buffer of the invention. Thus, the method of lysis can include any procedures that rely on a nucleic acid polymerase, including, but not limited to, PCR, sequencing, primer extension, cDNA synthesis (in a one-step or two-step protocol), and the like. Amplification reactions thus can be performed with any number of polymerases, including thermostable polymerases (e.g., Taq polymerase) and reverse transcriptases (e.g., MMTV RT). Amplification can amplify DNA and/or RNA, including, but not limited to isothermal methods.

Other non-limiting examples of enzymes and protocols that are compatible with the buffers of the invention include endonucleases and endonuclease cleavage of nucleic acids; exonucleases and exonuclease cleavage of nucleic acids (e.g., for degradation of RNA or DNA in a particular sample); ligases and ligation of two or more nucleic acids; kinases and phosphorylation of nucleic acids; phosphatases and dephosphorylation of nucleic acids; and other nucleic acid modifying enzymes.

Of course, these nucleic acid modification, analysis, or amplification procedures may be performed in a buffer of the invention, where the nucleic acids to be manipulated, analyzed, or amplified are provided without lysis of cells specifically using the buffer of the invention. That is, the present invention, while providing such manipulation, analysis, and amplification methods as part of the lysis method, also provides such methods independent of lysis of cells in a buffer of the invention. In some embodiments, ligases are used in combination with two or more short nucleic acids to detect target nucleic acids, including, but not limited to those having nucleotide polymorphisms (e.g., SNPs) and micro RNAs (miRNA).

In an embodiment, the method of lysing a cell comprises growing eukaryotic cells, providing 100,000 cells in a lysis vessel, adding 100 microliters (ul) of an RNase-free solution of 5 mM TCEP, 1% Triton X-100, pH 2.5, and vortexing for 1 minute to create a cell lysate composition. In particular embodiments, this method further comprises storing the resulting cell lysate composition at 4° C. As discussed above, the number of cells can range from 1 to 100,000, or even more. Thus, in various embodiments, the number of cells is from 1 to 100,000.

In a third aspect, the invention provides methods of amplifying one or more nucleic acids. In general, the method comprises exposing at least one nucleic acid to a composition of the invention, and amplifying the nucleic acid.

Exposing can be for any suitable period of time, but is typically a sufficient amount of time to permit the nucleic acid of interest to be in a form that is capable of serving as a template for amplification. Accordingly, the time can be as short as 1 second or less, or as long as a day or more. Exemplary times include, but are not limited to, those described with regard to lysis, above. Of course, the time will be dependent, at least to some extent, on the temperature at which the solution is maintained, with longer times generally being required at lower temperatures. Temperatures for exposing can be any temperatures that do not result in significant degradation of the nucleic acids (when taken in conjunction with the time of exposing). Thus, the temperature may be 4° C., 10° C., 22°-25° C., 30° C., 37° C., 42° C., 50° C., 65° C., 70° C., or any other suitable temperature.

In accordance with the disclosure above, exposing, in the method of amplifying at least one nucleic acid, can comprise lysing at least one cell containing the nucleic acid of interest. Likewise, exposing can comprise adding a nucleic acid of interest to a cell lysate, or lysing at least one cell containing the nucleic acid of interest and adding a second nucleic acid of interest to the composition. In view of the fact that exposing in this aspect of the invention can comprise lysing at least one cell, all of the considerations discussed above may be applicable to the exposing step according to the method of amplifying at least one nucleic acid.

The method of amplifying at least one nucleic acid comprises amplification of target nucleic acid(s). Numerous techniques for amplification of nucleic acids are known and widely practiced in the art, and any of those techniques are applicable according to the method of this invention. One of skill in the art may select the amplification method based on any number of considerations, including, but not limited to, speed, sensitivity, usefulness in amplifying a particular type of nucleic acid (e.g., RNA vs. DNA), and reliability.

Although the method may comprise isolation or purification (to at least some extent) of nucleic acids, it has surprisingly been discovered that amplification of target nucleic acids may be accomplished without purification of the nucleic acid beforehand. Thus, the cell lysate composition of the invention is suitable for direct nucleic acid amplification. In embodiments, amplifying is by a PCR technique. In certain embodiments, the PCR technique is QPCR or RTPCR (including quantitative RTPCR). For example, the FullVelocity™ (Stratagene) enzyme may be used to amplify nucleic acids provided by the lysis buffer of the invention.

Where one or more RNA are the nucleic acids of interest, the method may comprise a cDNA synthesis prior to, or at the time of, amplification. Numerous cDNA synthesis protocols are known in the art, and any suitable protocol may be used. For example, the Stratascript® First Strand Synthesis System from Stratagene may be used to prepare cDNA from RNA templates.

In certain embodiments, one or more control reactions are included, such as a control reaction to permit normalization of the amount of nucleic acid being amplified with respect to other amplification reactions that are being performed concurrently, or with respect to a standard amplification curve. Such control reactions can comprise adding one or more exogenous nucleic acids to the composition, and performing an amplification on that nucleic acid. The control reaction can alternatively comprise amplifying sequences present in nucleic acids naturally present in the composition, where such sequences have a known copy number and amplification efficiency. In other embodiments, control reactions for known sequences are performed in reaction vessels separate from the reaction vessel in which the amplification of interest is being performed. Various other control reactions are known and widely used for amplification reactions, and any of those control reactions may be included in the method of the present invention to determine the success and efficiency of one or more steps in the amplification process.

One control that is particularly notable in the context of the present invention is an internal control that permits the practitioner to evaluate, and thus normalize if desired, the number of cells present in a particular cell lysate sample. In this way, conclusions about the amount of target nucleic acid (e.g., a particular mRNA) in a sample may be made. More specifically, when comparing amplification results of two different samples of cells, it is often not possible to determine with a high degree of accuracy, the number of cells in the original sample, the number of cells successfully lysed, or the total amount of nucleic acid liberated from each sample. Thus, accurate comparisons of the total amount of a target nucleic acid in different samples (e.g., mRNA representing an expressed gene) is not possible. Currently, housekeeping genes or rRNA species are used as markers to standardize or normalize samples from different cells or tissues. However, the currently used internal standards have been reported to be inconsistent, and thus do not provide the accuracy and repeatability that is needed for an internal control.

An internal control that is standardized among different samples and cell types is thus a desirable feature of a PCR protocol. The present invention, in embodiments, encompasses such an internal control by including within the compositions, methods, and kits, primers that are specific for unique sequences on one or more of the chromosomes of a given cell. Because these unique genomic sequences are present in only one copy per haploid genome (i.e., two copies per cell), they can be used to prepare a standard curve for a particular amplification procedure. Inclusion of the primers in the amplification reactions for test samples (either in the same reaction vessel or in a second reaction vessel comprising the same components) results in an amplification curve for the unique genomic sequences. These curves can be compared to the standard curve for each primer set, and the amount of nucleic acid, and thus the number of cells in the original sample, can be calculated. With this knowledge, the amount of a target nucleic acid in numerous different samples can be determined, and accurately compared with other samples.

In a fourth aspect, the invention provides methods of stabilizing at least one nucleic acid. In general, the methods comprise exposing at least one nucleic acid to a composition of the invention and maintaining the newly formed composition for a period of time. It has surprisingly been found that the compositions of the invention can be used to store nucleic acids for relatively long periods of time without significant loss of nucleic acid or nucleic acid quality. Thus, nucleic acids can be stored for relatively long periods of time and subsequently used for analysis, such as by a PCR technique.

Exposing in this aspect of the invention, will be in accordance with many of the considerations discussed above with regard to cell lysis and amplification. Substances in addition to TCEP and non-ionic detergent(s), times of exposing, temperatures, and other parameters may vary, but should be selected with consideration of the ultimate use for the nucleic acids, and in recognition of the effects of the various parameters, alone and in combination with each of the others and the combination as a whole, on nucleic acid stability. Having noted this, however, it is to be understood that various combinations of parameters, some of which are detailed in the Examples below, have been tested, and the compositions of the invention have been found to be well suited for long-term, stable storage of nucleic acids.

In general, nucleic acids, and RNA in particular, has been found to be somewhat unstable when stored above freezing. Likewise, it has proved to be unstable when exposed to repeated freeze-thaw cycles. Thus, nucleic acid samples for further processing (e.g., PCR amplification) are typically used fresh (i.e., immediately upon isolation), or stored frozen and used immediately upon thawing, without attempting to re-freeze and re-use the sample. The present invention permits one to store nucleic acids for extended periods of time without degradation. Thus, one need not use the nucleic acids obtained in a lysis protocol immediately, and need not store those nucleic acids frozen until ready for use. Rather, lysed cells or purified nucleic acids may be maintained at relatively high temperatures for relatively long periods of time before use in analysis protocols. For example, a cell lysate comprising a composition according to the present invention may be stored at 4° C. for 16 hours or more before an amplification reaction is performed on the nucleic acids in the composition.

As mentioned above, the compositions of the invention permit one to maintain nucleic acids in a stable state for extended periods of time. The times can be short, such as 1 minute or less. Alternatively, the times may be long, such as days or weeks. For example, a sample containing at least one nucleic acid (whether it be a purified or semi-purified nucleic acid, or one that is present in a complex mixture, such as a cell lysate), can be maintained without significant degradation of the nucleic acid for 5 minutes, 10 minutes, 30 minutes, 60 minutes, 2 hours, 3 hours, 4 hours, 5 hours, 6 hours, 7 hours, 8 hours, 9 hours, 10 hours, 12 hours, 15 hours, 20 hours, one day, 36 hours, 2 days, 3 days, 4 days, 5 days, 6 days, a week, two weeks, five weeks, ten weeks, twenty weeks, twenty-five weeks, or twenty-six weeks (one-half year) or more, or any fraction of these times. In an exemplary embodiment, nucleic acids are maintained in a stable state for at least 25 days at 4° C. In another exemplary embodiment, nucleic acids are maintained in a stable state for at least 168 days at room temperature. The present invention provides for stable storage of nucleic acids at temperatures up to about room temperature (i.e., up to about 25° C.) for one year or more. As will be understood by those of skill in the art, an undisturbed sample of nucleic acids that is stable at a given temperature above freezing for more than 48 hours is highly stable indefinitely. Accordingly, the present invention provides a method and composition for stable maintenance of at least one nucleic acid.

As used herein, “significant degradation” is meant to describe degradation that renders a nucleic acid unsuitable for a chosen purpose. Thus, if the purpose is amplification of a particular sequence by PCR, then significant degradation would render that sequence of the nucleic acid unsuitable for amplification by PCR, regardless of whether it is still suitable for other purposes, such as for amplification of another sequence on the same nucleic acid. Thus, a loss of suitability for one purpose does not necessarily render the nucleic acid unsuitable for all purposes.

As mentioned above, the compositions of the invention permit one to maintain nucleic acids in a stable state at various different temperatures. The temperature can be any temperature selected by the practitioner, and which is suitable and compatible for use of the nucleic acid for a subsequent purpose. Thus, the temperature may be quite low, for example −80° C. (e.g., frozen in liquid nitrogen or dry ice) or −20° C. (e.g., in a standard laboratory freezer). It can likewise be above freezing, but relatively cool, for example 4° C. or 10° C. In addition, it can be higher, such as at ambient room temperature (e.g., 20° C.-25° C.).

Compositions of the invention may also be reaction mixtures. Thus, they may be exposed to higher temperatures, such as, for example, 30° C., 37° C., 42° C., 50° C., 62° C., and 72° C. The present invention permits relatively long storage of nucleic acids at these higher temperatures. Accordingly, the present invention provides a method and composition for stable maintenance of at least one nucleic acid at various temperatures. In view of the fact that the methods and compositions of the invention permit stable maintenance of nucleic acids for extended periods of time and at various temperatures, the present invention encompasses any suitable combination of time and temperature for maintaining nucleic acids.

In embodiments, the methods of stabilizing a nucleic acid include exposing a cell containing the nucleic acid to a composition of the invention for a sufficient amount of time for lysis of the cell to occur, then maintaining the cell lysate, or a fraction of the cell lysate containing nucleic acids, for a period of time prior to use of the nucleic acids. In certain embodiments, the stabilized nucleic acid is subsequently used for amplification, such as by a PCR technique.

In a fifth aspect, the invention provides kits. In general, the kits contain a composition of the invention. The kits can further comprise one or more substances, materials, reagents, etc. that can be used for lysis of cells, storage of nucleic acids or cell lysates, or manipulation or analysis of nucleic acids, such as amplification of nucleic acids. In embodiments, some or all of the materials, reagents, etc. necessary to lyse cells, amplify nucleic acids, and/or purify nucleic acids are included in the kit.

For example, a kit may contain a container holding a buffer comprising TCEP and at least one non-ionic detergent, and, in the same or a separate container, at least one reagent for amplification of a target nucleic acid. Thus, it can comprise at least one primer, such as two primers, for amplification of a target nucleic acid. It also may include at least one other primer for amplification of a target nucleic acid, which can be, but is not necessarily, the same nucleic acid (and even the same sequence within the same nucleic acid) that is the target for one or more other primer(s) in the kit. In some embodiments, the kits comprise betaine. In some embodiments, the kits comprise two or more primers for amplifying one or more unique genomic sequences.

Viewed from another perspective, a kit of the present invention may be a kit for analysis of nucleic acids, further comprising a composition comprising TCEP and a non-ionic detergent. Thus, for example, it can be a kit for detection of RNA using a one-step QRT-PCR, such as the Brilliant® QRT-PCR kit from Stratagene or the Full Velocity® QRT-PCR kit from Stratagene, further comprising at least one container containing a buffer comprising TCEP and a non-ionic detergent. Such kits can comprise, in packaged combination, at least one reverse transcriptase, at least one DNA polymerase, such as Taq DNA polymerase, Pfu DNA polymerase, Pfx DNA polymerase, Tli DNA polymerase, Tfl DNA polymerase, and klenow, an RNase inhibitor, nucleotides (e.g., any or all of the four common deoxynucleotides), primers, probes, or labels (such as, for example, SYBR green), or any combination of two or more of these. Alternatively, it can be a kit that can be used for detection of RNA using a two-step QRT-PCR, such as the StrataScript® First Strand cDNA synthesis kit (which can be used in conjunction with other kits and protocols, such as those mentioned above). Alternatively, the kit can be one that can be used for detection of DNA using a PCR technique, such as QPCR. In addition, the kit can be one that is used for detection of short interfering RNA (siRNA). In embodiments, the kits comprise transfection or transformation reagents.

The kits can comprise the components in a single package or in more than one package within the same kit. Where more than one package is included within a kit, each package can independently contain a single component or multiple components, in any suitable combination. As used herein, a combination of two or more packages or containers in a single kit is referred to as “in packaged combination”. The kits and containers within the kits can be fabricated with any known material. For example, the kits themselves can be made of a plastic material or cardboard. The containers that hold the components can be, for example, a plastic material or glass. Different containers within one kit can be made of different materials. In embodiments, the kit can contain another kit within it. For example, the kit of the invention can comprise a kit for purifying nucleic acids.

The kit of the invention can comprise one or more components useful for amplifying target sequences. In embodiments, some or all of the reagents and supplies necessary for performing PCR are included in the kit. In exemplary embodiments, some or all reagents and supplies for performing QPCR are included in the kit. In other exemplary embodiments, some or all reagents and supplies for performing RT-PCR are included in the kit. Non-limiting examples of reagents are buffers (e.g., a buffer containing Tris, HEPES, and the like), salts, and a template-dependent nucleic acid extending enzyme (such as a thermostable enzyme, such as Taq polymerase), a buffer suitable for activity of the enzyme, and additional reagents needed by the enzyme, such as dNTPs, dUTP, and/or a UDG enzyme. In embodiments, the kit comprises Brilliant® SYBR® Green QPCR Master Mix (Catalog # 600548, Stratagene, La Jolla, Calif.). A non-limiting example of supplies is reaction vessels (e.g., microfuge tubes).

The kit can comprise at least one dye for detecting nucleic acids, including, but not limited to, dsDNA. In embodiments, the kit comprises a sequence-non-specific dye that detects dsDNA, such as SYBR® Green dye (Molecular Probes, Eugene, Oreg.). The dye is preferably contained alone in a container. In embodiments, the dye is provided as a concentrated stock solution, for example, as a 50× solution. In embodiments, the kit comprises a passive reference dye. In these embodiments, the passive reference dye can be included in the kit alone in a separate container. The passive reference dye can be provided as a concentrated stock solution, for example, as a 1 mM stock solution. A non-exclusive exemplary passive reference dye is ROX dye. In embodiments, the kit contains either a DNA-detecting dye or a passive reference dye. In other embodiments, the kit contains both a DNA-detecting dye and a passive reference dye.

The kit can also comprise one or more components useful for purifying nucleic acids. In embodiments, these components are particularly suited for purifying target nucleic acids from eukaryotic cell cultures. The components can be, among other things, reagents and supplies that can be used to purify nucleic acids. Non-limiting examples of such reagents and supplies include, but are not limited to, a DNA binding solution, a wash buffer, and containers, such as microfuge tubes, for collection of binding solutions, wash buffers, and purified nucleic acids. The components can also contain a resin, gel, or other substance that is useful for purifying nucleic acids. In embodiments, the kit comprises the components of the StrataPrep® PCR Purification Kit (Catalog # 400771, Stratagene, La Jolla, Calif.).

The invention, in general, is suitable for use in both research and diagnostics. That is, the compositions and methods of the invention can be used for the purpose of identifying various nucleic acids or expressed genes, or for other research purposes. Likewise, the compositions and methods can be used to diagnose numerous diseases or disorders of humans and animals. In addition, they can be used to identify diseased or otherwise tainted food products (e.g., foods that are infected with one or more pathogenic organisms), or the presence of toxic substances or toxin-producing organisms in a sample. Thus, the compositions and methods have human health and veterinary applications, as well as food testing and homeland security applications.

### EXAMPLES

The invention will be further explained by the following Examples, which are intended to be purely exemplary of the invention, and should not be considered as limiting the invention in any way.

### Example 1

### Preparation of a Buffer of the Invention

A composition comprising Tris(2-carboxyethyl)phospine (TCEP; MW 286) and Triton X-100 was made for use in further examples. 0.038 g TCEP was dissolved into 26 ml of RNase-free water. To this, 50 ul of 0.5 M HCl was added to adjust the pH to 2.5. To the TCEP solution, 260 ul of 100% Triton X-100 was added, and the final solution mixed until a homogeneous solution was obtained. The final solution comprised 5 mM TCEP and 1% Triton X-100 at a pH of 2.5. The composition was stored at 4° C. until use.

### Example 2

### Cell Lysis

Jurkat cells were grown in RPMI medium in accordance with standard cell culture techniques. The culture was split 2:1 into new RPMI medium, and 2 hours later, cells were resuspended in PBS at a concentration of 1,00,000 cells per ml. 100 ul of cells were aliquotted into new tubes and centrifuged at 1000×g for 5 minutes to pellet the cells. The PBS was aspirated and 100 ul of lysis buffer from Example 1 was added. The cells were lysed by vortexing for 1 minute, then placed on ice or stored at 4° C. or frozen (−20° C. or −80° C.) until use. To test whether heating is required to inactivate enzymes present in the composition (which it is not), the cell lysate was heated at 65° C. for 10 minutes.

### Example 3

### cDNA Synthesis

cDNA was synthesized from RNA present in the cell lysate generated in Example 2 using the StrataScript® First Strand Synthesis kit from Stratagene, as follows: To 28 ul of lysate, 1 ul of either water or control RNA (1.8 ng/ul), and 3 ul of random primers (100 ng/ul) were added and thoroughly mixed. The mixture was incubated at 70° C. for 5 minutes, then cooled to room temperature for 10 minutes. Meanwhile, a cocktail of the following reagents was made: 4 ul of the first strand buffer, 1 ul of RNase Block (40 U/ul), 2 ul of 100 mM dNTPs, and 1 ul of StrataScript® RT (200 U/ul) or 1 ul water (for a no RT control reaction).

The 8 ul cocktail was added to the 32 ul cell lysate, and the reaction mixture incubated at 42° C. for one hour for cDNA first strand synthesis. The reaction mixture was then incubated at 90° C. for 5 minutes to inactivate the RT.

The first strand synthesis mixture was stored at 4° C. until use, or, alternatively, used immediately for amplification.

### Example 4

### Amplification of cDNA

The first strand synthesis reaction mixture obtained by the procedure of Example 3 was subjected to amplification to detect a nucleic acid sequence of interest. More specifically, 5 ul of the heat-inactivated 40 ul reaction mixture (representing approximately 3500 cells) was combined and thoroughly mixed with the following cocktail: 5 ul of 10 Pfu buffer, 0.4 ul of 100 mM dNTPs, 2 ul of β-actin primers (Stratagene Cat. No. 302010), 1 ul of Pfu turbo hotstart DNA polymerase (2.5 U/ul), and 36.6 ul water. In addition, 3500 cells were processed according to the Cells-to-Signal™ (Ambion) protocol using the Cells-to-Signal™ buffer. Likewise, 3500 cells were processed according to the procedure above, with the exception that a buffer comprising 5 mM salicylic acid and 1% Triton X-100 or comprising 1% Triton X-100 in PBS, instead of a buffer of the present invention, was used. As a positive control, 5.4 ng of purified Jurkat RNA was used.

Each mixture was amplified in a Stratagene RoboCycler® using the following program: 1^{st} cycle −95° C. for 2 min; 2^{nd}-36^{th} cycle −95° for 1 min, 60° C. for 1 min, 72° C. for 1 min; 37^{th} cycle −72° C. for 1 min. The reaction mixture was loaded on an agarose gel, and a band representing amplified β-actin sequences was obtained.

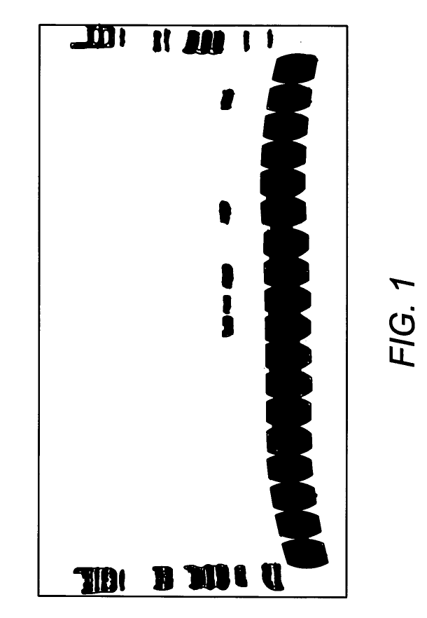

The results of the amplification reactions are presented in FIG. 1. In this figure, the lanes (from left to right) contain amplification reactions from various protocols, as follows: 1) molecular weight marker; 2) 3500 cells processed using the Ambion Cells-to-Signal™ protocol without heating; 3) 3500 cells processed using the Ambion Cells-to-Signal™ protocol with heating; 4) 3500 cells plus 5.4 ng of purified Jurkat RNA, processed using the Ambion protocol; 5) no cells, processed using the Ambion system (negative control); 6) 3500 cells processed according to a protocol of the present invention, but using a buffer comprising 5 mM salicylic acid and 1% Triton X-100 instead of a buffer according to the invention, without heating; 7) 3500 cells processed according to a protocol of the present invention, but using a buffer comprising 5 mM salicylic acid and 1% Triton X-100, with heating; 8) 3500 cells plus 5.4 ng of purified Jurkat RNA, suspended in 5 mM salicylic acid and 1% Triton X-100, and processed according to a method of the invention; 9) no cells, processed using the salicylic acid/Triton composition and the method of the present invention (negative control); 10) 3500 cells processed according to a protocol of the present invention, using a buffer and protocol according to the invention, without heating; 11) 3500 cells processed according to a protocol of the present invention, using a buffer according to the invention, with heating; 12) 3500 cells plus 5.4 ng of purified Jurkat RNA, suspended in a buffer according to the present invention and processed according to a protocol of the invention; 13) no cells, processed using a buffer and protocol of the present invention (negative control); 14) 3500 cells processed according to a protocol of the present invention, but using a buffer comprising PBS and 1% Triton X-100 instead of a buffer according to the invention, without heating; 15) 3500 cells processed according to a protocol of the present invention, but using a buffer comprising PBS and 1% Triton X-100, with heating; 16) 3500 cells plus 5.4 ng of purified Jurkat RNA, suspended in PBS and 1% Triton X-100, and processed according to a method of the invention; 17) no cells, processed using the PBS/Triton composition and the method of the present invention (negative control); 18) 5.4 ng of purified Jurkat RNA, suspended in water; 19) no cells or purified RNA (negative control); 20) molecular weight markers.

The results presented in FIG. 1 show that a buffer according to the present invention provides a rapid and sensitive buffer for lysis of cells and amplification of target nucleic acids. It also shows that lysis and amplification provides specific amplification of target nucleic acids, which is superior to a commercially available lysis and amplification buffer. More specifically, lanes 10-13, which show that the buffer of the invention successfully permitted cell lysis, cDNA synthesis, and cDNA amplification. Lysis and amplification was possible without heating, and did not occur in the absence of cells or RNA. While some amplification was seen in the commercial product, the amount of amplification was much less than that seen using the buffer and protocol of the present invention.

### Example 5

### Stability of Nucleic Acids Stored in a Buffer of the Invention

To determine the effects of the compositions of the invention on nucleic acid stability, cell lysates were maintained at various temperatures for various lengths of time, then analyzed for the presence and amount of target sequences, and the general quality of nucleic acids in the samples. The results show that target sequences can be amplified essentially to the same extent whether freshly obtained or after storage for up to 3 weeks. Furthermore, amplification of target sequences is more sensitive in a lysis and amplification buffer of the present invention that in a lysis and amplification buffer presently available commercially.

HeLa cells were grown on DMEM media as per standard cell culture protocols. Cells were harvested by low speed centrifugation (1,000×g), and washed with PBS, then resuspended in PBS at a concentration of approximately 1,000,000 per ml. For each reaction, 10,000 cells were aliquotted into different 1.5 ml microcentrifuge tubes and pelleted by centrifugation at 1,000×g for 5 minutes. PBS was removed by aspiration, then 100 ul of the lysis buffer according to Example 1 was added to each pellet, and the composition vortexed for 1 minute. The resulting cell lysates were stored at 4° C., −20° C., or −80° C. for various times.

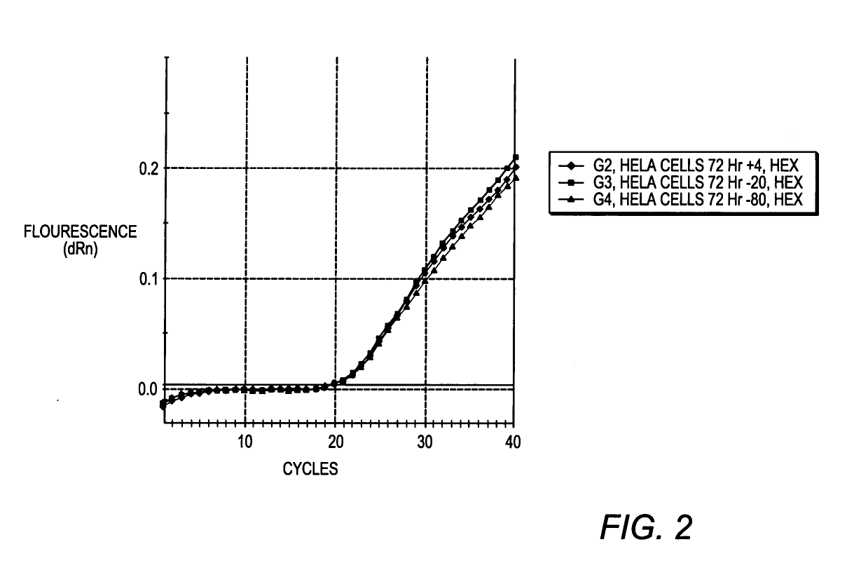

Cell lysates were removed from storage after approximately 72 hours and assayed for the presence of a target nucleic acid sequence using QRT-PCR. The target was the human GAPD (GAPDH) endogenous control from Applied Biosystems (Cat# 4326317E; (VIC/MGB Probe, Primer Limited)). As can be seen from FIG. 2, there is no significant loss in target sequence depending on the temperature at which the cell lysates are stored. That is, the sample stored at 4° C. has substantially the same amplification profile as the samples stored at −20° C. and −80° C. Thus, the buffer of the invention provides for stable storage of nucleic acids at various temperatures, including temperatures above 0° C.

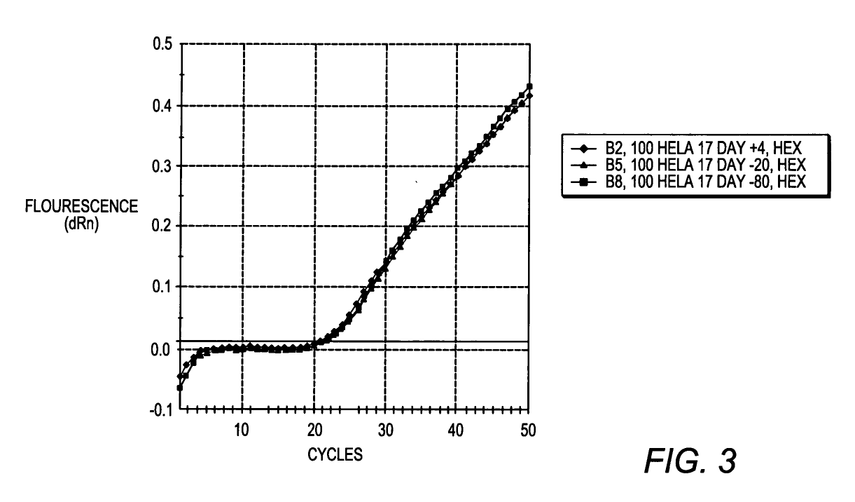

Cell lysates were also removed from storage after 17 days and assayed for the presence of the same target nucleic acid sequence as assayed at 72 hours. The results are depicted in FIG. 3, which shows that, in a similar manner to that seen in FIG. 1, there is no significant loss in target sequence depending on the temperature at which the cell lysates are stored. Furthermore, when the data presented in FIG. 2 is compared to that in FIG. 1, it can be seen that there is no significant loss in target sequence when samples are stored up to 17 days at various temperatures (comparing Ct values between the two figures).

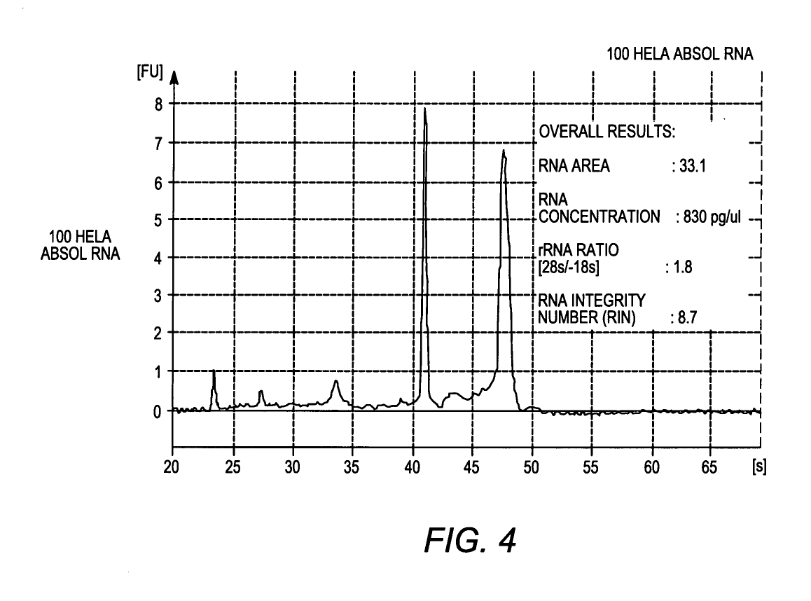

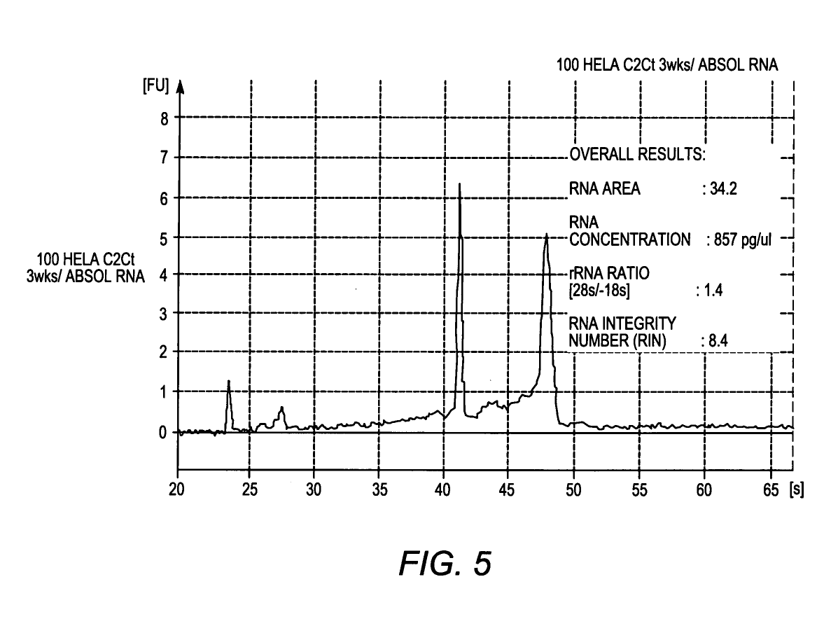

To further characterize the quality of nucleic acids obtained by the lysis method of the invention, and to further characterize the stability of the nucleic acids in a buffer of the invention over time, capillary electrophoresis of RNA obtained using a buffer of the invention (see Example 1) was performed using RNA obtained using the Absolutely RNA® kit (Stratagene), either directly after cell lysis (FIG. 4) or after storage of cell lysates at −20° C. for 3 weeks (FIG. 5). The electropherograms presented in FIGS. 4 and 5 show that there is no significant degradation of RNA in samples stored at −20° C. for at least 3 weeks as compared to freshly-isolated RNA. Accordingly, these two figures show that the buffer of the present invention is suitable for long-term, stable storage of cell lysates and nucleic acids.

To yet further characterize the quality of the nucleic acids obtained by the lysis method of the invention, and to yet further characterize the stability of the nucleic acids in a buffer of the invention over time, Jurkat cells were lysed with a buffer of the invention comprising 5 mM TCEP and 1% Triton X-100, pH 2.5, then either assayed immediately for expression of GADPH in a TaqMan® QRT-PCR amplification, or stored at room temperature (22° C.-25° C.) for 1 hour, 2 hours, 4 hours, 8 hours, 26 hours, or 168 hours. For each PCR reaction, cell lysates corresponding to 200 Jurkat cells were used.

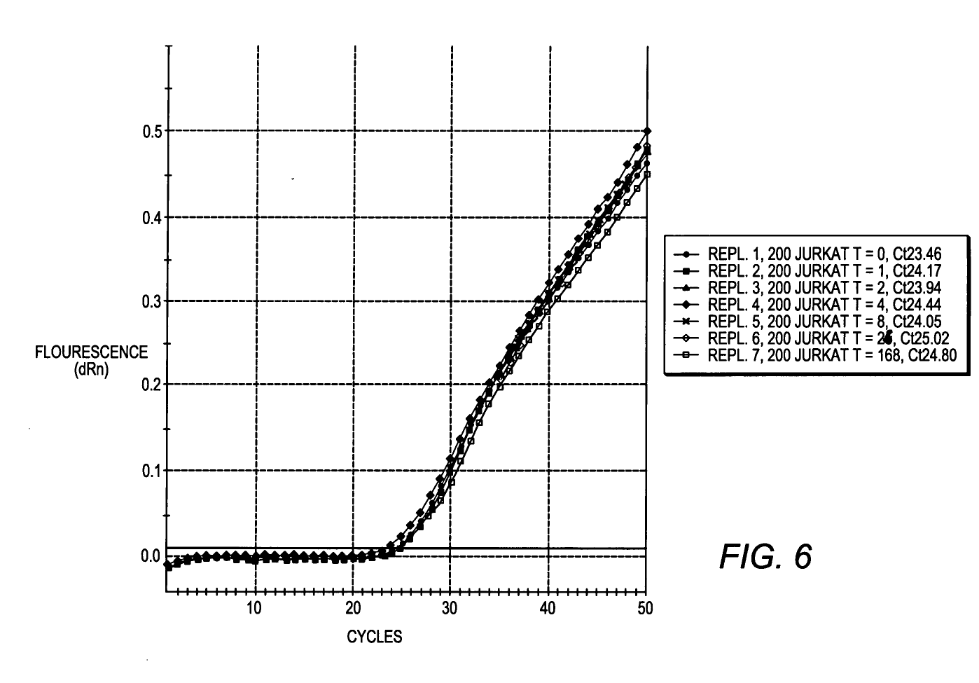

The results of the room temperature time-course stability study is depicted in FIG. 6. The figure shows that the buffer is suitable not only for lysis of eukaryotic cells and amplification of nucleic acid released from the cells, but also for long-term stable storage of the nucleic acids, including mRNA, at room temperature for at least 168 hours. Indeed, the figure shows no significant degradation of target mRNA, as determined by amplification efficiency, between samples freshly obtained from cell lysis and those stored at room temperature for 168 hours (one week). Thus, buffers of the invention may be used to stably store nucleic acid samples, including complex samples such as cell lysates, at temperatures from about 25° C. to about −80° C. for hours days, or weeks. This is a significant improvement over one-step lysis and amplification buffers currently available in the art.

### Example 6

### Standard Curve for Use as an Internal Control to Normalize Starting Material

To provide a basis for normalization of the amount of starting materials (cells) from sample to sample when using the buffers and protocols of the invention, a standard curve for amplification of human chromosome 9 was developed. A primer set (2 primers) that is specific for a unique sequence on human chromosome 9 was designed, having the following sequences:

| | |
|---|---|
| chr9 up: | |
| 5′-TATAAGAAACTACTAAGCACCCAAAGG-3′; | (SEQ ID NO:1) |
| chr9 down: | |
| 5′-AAGAAAGGAGTCTAAGTGACTCAACAG-3′. | (SEQ ID NO:2) |

Design of the primers was in accordance with the procedure disclosed in a co-pending application filed on the same day as the present application, under Attorney Docket No. STG-116, which is hereby incorporated herein by reference.

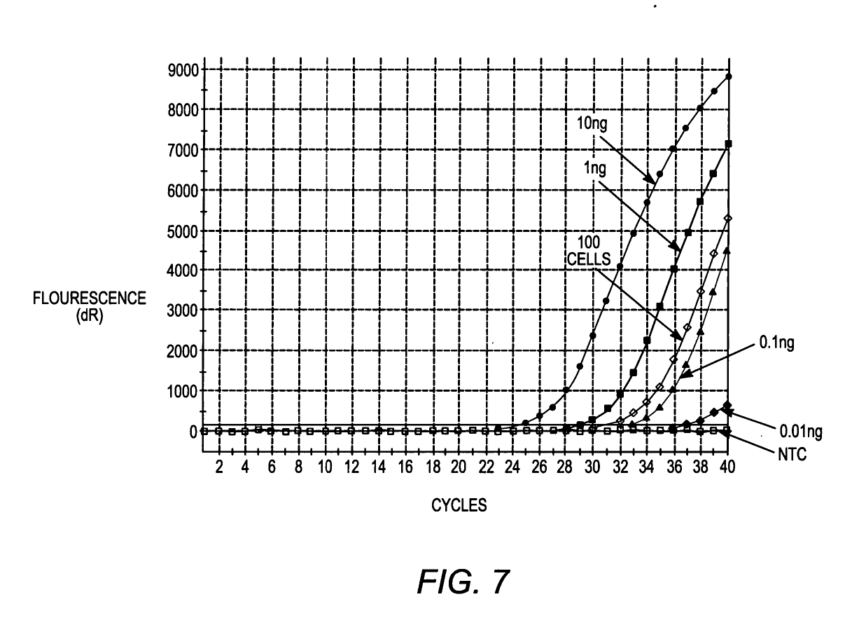

QPCR was performed using the FullVelocity™ SYBR® Green QPCR Master Mix and protocol (Stratagene) and different amounts of human DNA (0.01 ng, 0.1 ng, 1 ng, and 10 ng). QRT-PCR was performed using a buffer of the invention, and 100 HeLa cells. Amplification plots of the various reactions are depicted in FIG. 7.

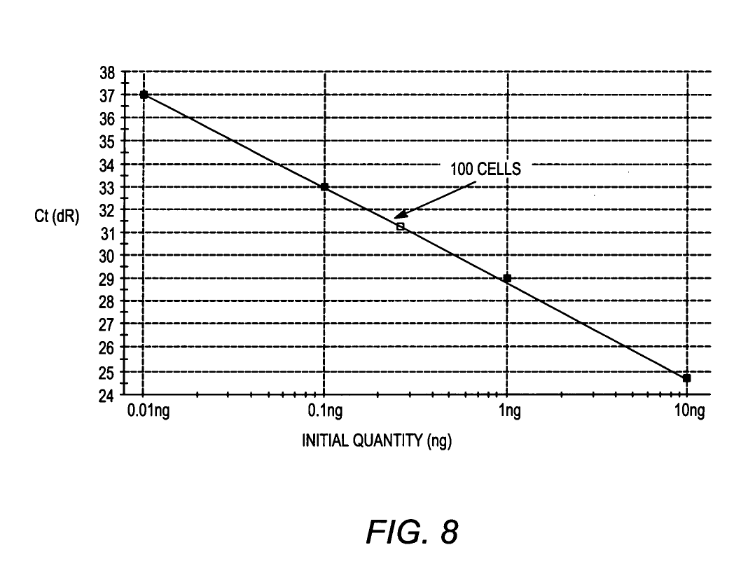

The Ct values for each amount of genomic DNA tested were plotted, along with the Ct value obtained for 100 cells, on a graph of Ct vs. initial quantity, and a linear standard curve obtained. The standard curve for this experiment is depicted in FIG. 8, and shows that the Ct value obtained for lysis and amplification of 100 cells falls directly on the curve at the point representing approximately 0.25 ng of initial genomic DNA.

### Example 7

### Amplification of Unique Genomic DNA in Cell Lysates Created with a Buffer of the Invention

The concept that the one-step lysis and amplification buffer of the invention can be used to effectively and quantitatively amplify unique genomic sequences, and thus that unique genomic sequences can serve as an internal control and normalization factor for amplification of target sequences with the buffer of the invention, was further validated by lysis of HeLa cells and amplification of a unique genomic sequence using the primers disclosed above (SEQ ID NO: 1 and SEQ ID NO:2).

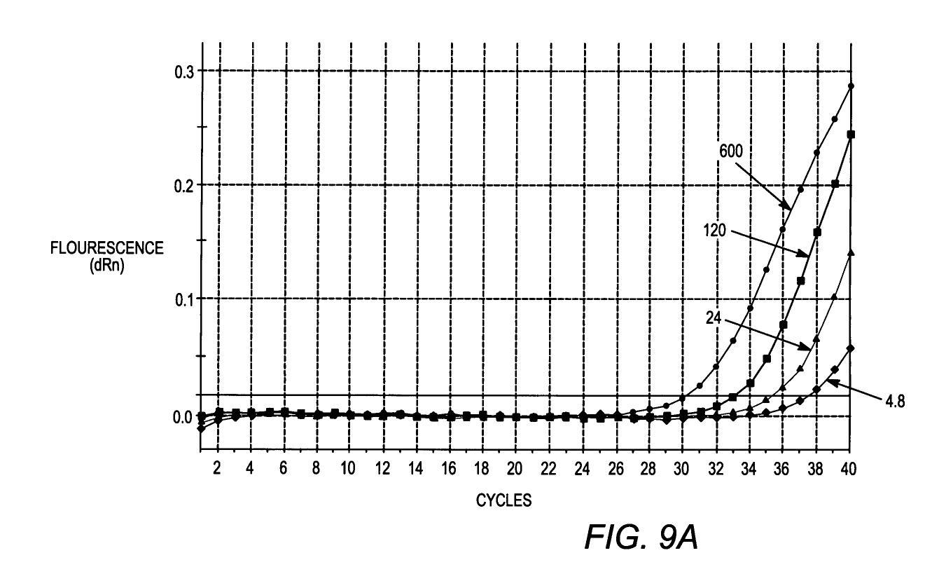

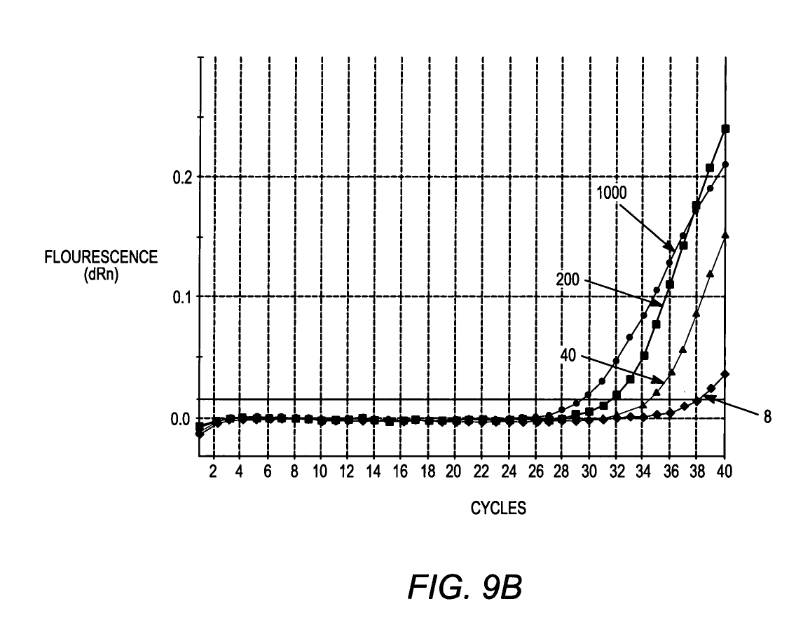

The amplification reactions comprised either 3 ul of HeLa cell lysate in a one-step buffer comprising 5 mM TCEP and 1% Triton X-100, pH 2.5, or 5 ul of cell lysate in that one-step buffer. The number of cells represented by the cell lysate samples ranged from 4.8 cells to 1,000 cells. The results of the amplification reactions are depicted in FIGS. 9A and 9B.

More specifically, FIG. 9A depicts the amplification plots of cell lysates created in a buffer comprising 5 mM TCEP, 1% Triton X-100, pH 2.5, where the plots represent amplification of cell lysates from 4.8 cells, 24 cells, 120 cells, and 600 cells. FIG. 9B depicts the amplification plots of cell lysates created in the same buffer, where the plots represent amplification of cell lysates from 8 cells, 40 cells, 200 cells, and 1,000 cells.

The results presented in FIGS. 9A and 9B show that amplification of gDNA in these two buffers can be accomplished using a primer set that specifically amplifies a unique genomic sequence in the cell of interest, from which the sample tested originates. The results also indicate that amplification profiles vary linearly with amount of genomic nucleic acid supplied in cell lysates resulting from lysis of cells using the “one-step” buffer of the invention. In addition, the data shows that the amount of cells from which a cell lysate is obtained can be determined using the unique genomic sequences and corresponding amplification primers, when used in conjunction with a buffer of the present invention. Accordingly, cell samples can be normalized for amounts of starting materials, and valid, accurate conclusions regarding the absolute or relative amounts of various nucleic acid targets (e.g., expression products) may be drawn.

### Example 8

### Lysis of Cheek Cells and Amplification of Liberated RNA

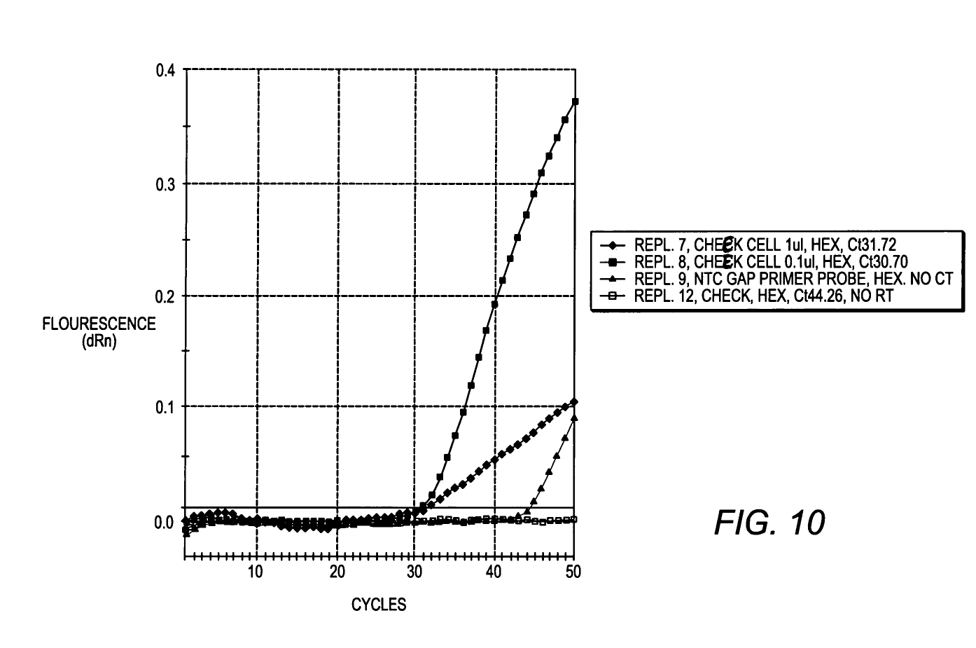

The applicability of a buffer of the invention for lysis of freshly isolated human cheek cells (mouth mucosal cells) and detection of RNA from those cells was tested. The procedure was as follows: subjects thoroughly rinsed their mouths with sterile water; a sterile swab was run over the interior of each subject's cheek six times; the swab was placed in 100 ul of a buffer comprising 5 mM TCEP and 1% Triton X-100, pH 2.5, and agitated briefly to dislodge cells from the swab; and the resulting composition was vortexed 1 minute to lyse cells. QRT-PCR reactions were performed using 1 ul and 0.1 ul of the vortexed composition. The QRT-PCR reactions were performed using the Stratagene Brilliant QRT-PCR kit and TaqMan GAP primer/probe sets. A control reaction lacking nucleic acid (“no template control” or “NTC”) and a control reaction lacking reverse transcriptase (“No RT”) were performed as well. The results of the amplification reactions are presented in FIG. 10.

As can be seen from FIG. 10, the buffer of the invention was suitable for amplification of as little as 0.1 ul of the cell lysate. Furthermore, amplification of both 1 ul and 0.1 ul yielded Ct values of about 31-32, well below the Ct values for the negative controls. Thus, FIG. 10 shows that buffers according to the invention can be used to assay not only freshly isolated cells from patients, but small amounts of cells as well. The diagnostic potential of the methods, compositions, and kits of the invention are evident from this experiment.

### Example 9

### Lysis of Cells in Whole Blood and Detection of Nucleic Acids Liberated from those Cells

To further show the applicability of the present buffers, methods, and kits of the invention for diagnostic and research purposes, whole blood was exposed to a buffer of the invention, and the resulting cell lysate analyzed for the presence of target nucleic acid sequences. The procedure for lysis of cells and detection of nucleic acids was as follows: 8 ul of a subject's blood from a finger stick was added to 100 ul of a buffer of the invention comprising 5 mM TCEP and 1% Triton X-100, pH 2.5; and the composition was vortexed for 1 minute to create a lysate. QRT-PCR reactions were performed on 1 ul and 0.1 ul of the lysate, using the Stratagene Brilliant QRT-PCR kit and TaqMan GAP primer/probe sets. A no template control (NTC) and no reverse transcriptase control (No RT) were included.

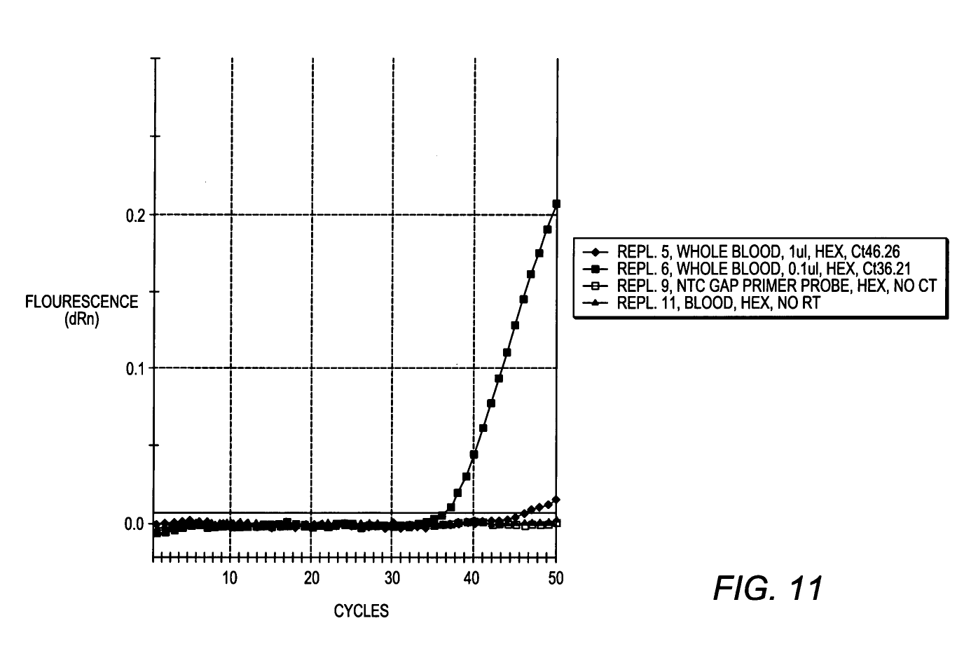

As can be seen from FIG. 11, 1 ul of the whole blood lysate provided a satisfactory amplification profile, with a Ct of about 36. Under the conditions used, the NTC and No RT control reactions did not produce an amplification profile. FIG. 11 thus shows that the buffers of the invention can be used to detect target sequences in cells present in a complex mixture such as whole blood, without the need to first isolate, purify, or otherwise separate the target cells or target nucleic acid sequences from the whole blood environment. It thus shows the applicability of the present invention in diagnostic areas as well as research areas.

### Example 10

### Lysis of Mouse Liver Cells and Amplification of Liberated RNA

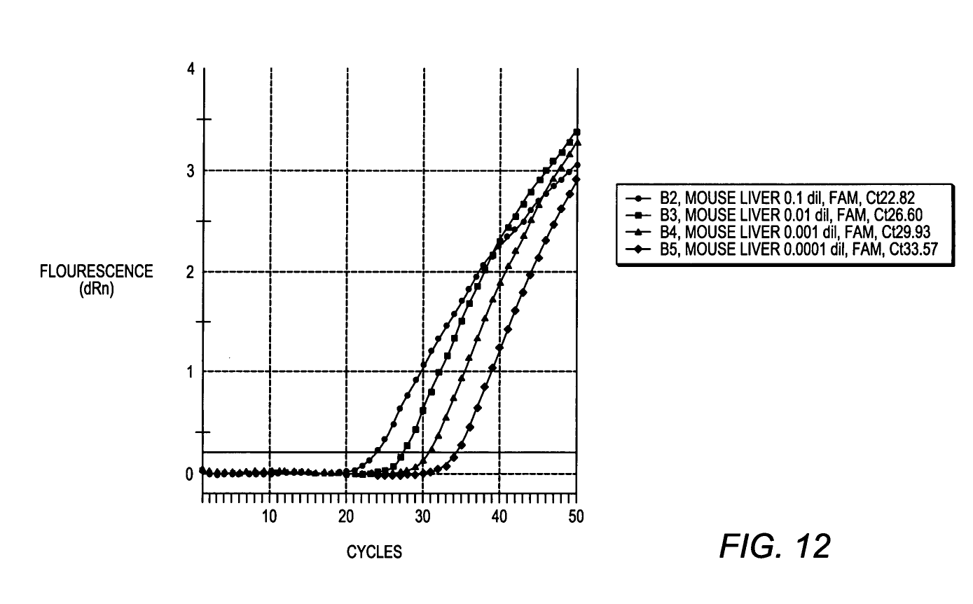

The applicability of a buffer of the invention for lysis of mouse liver cells in a homgenate and detection of RNA from those cells was tested. The procedure was as follows: 40 mg of mouse liver was combined with 100 ul of a buffer comprising 5 mM TCEP and 1% Triton X-100, pH 2.5, and the liver tissue was homogenized completely; and the resulting composition was vortexed 1 minute to lyse cells. QRT-PCR reactions were performed using 0.1 ul and 0.01 ul, 0.001 ul, and 0.0001 ul of the vortexed composition. The QRT-PCR reactions were performed using the Stratagene Brilliant QRT-PCR kit and TaqMan GAP primer/probe sets. The results of the amplification reactions are presented in FIG. 12.

As can be seen from FIG. 12, the buffer of the invention was suitable for amplification of as little as 0.0001 ul of the cell lysate. Thus, FIG. 12 shows that buffers according to the invention can be used to assay cells and specific nucleic acid targets in complex mixtures, such as those of organ homogenates. The diagnostic and research potentials of the methods, compositions, and kits of the invention are evident from this experiment.

### Example 11

### Comparison of Amplification Between Purified RNA and RNA Obtained Directly from Cells Using a Buffer of the Invention

To help determine the efficiency of the buffers of the invention in detecting target nucleic acids, experiments were designed to compare amplification of a target nucleic acid sequence (GAP) on purified mRNA and mRNA present in cell lysates created using a buffer of the invention. More specifically, 100,000 HeLa cells were harvested in two separate microcentrifuge tubes (100,000 cells per tube). To each cell pellet, 100 ul of a buffer comprising 5 mM TCEP and 1% Triton X-100, pH 2.5, was added, and the resulting composition vortexed for 1 minute to resuspend and lyse the cells. From one tube, mRNA was isolated using the Absolute mRNA magnetic bead isolation kit from Stratagene, according to the manufacturer's instructions, and mRNA eluted from the beads in 100 ul of sterile water. Samples of the eluate were assayed for GAP sequences using the Stratagene Brilliant QRT-PCR kit and TaqMan GAP primer/probe sets. The samples contained 1 ul, 0.1 ul, 0.01 ul, or 0.001 ul of the eluate per reaction. From the second tube, cell lysate was used directly for PCR reactions. Samples of the lysate were assayed for GAP sequences using the Stratagene Brilliant QRT-PCR kit and TaqMan GAP primer/probe sets, as for the other tube. The samples contained 1 ul, 0.1 ul, 0.01 ul, or 0.001 ul of the eluate per reaction. The amount of sample used from each of the two tubes for each dilution corresponded to the same number of cells.

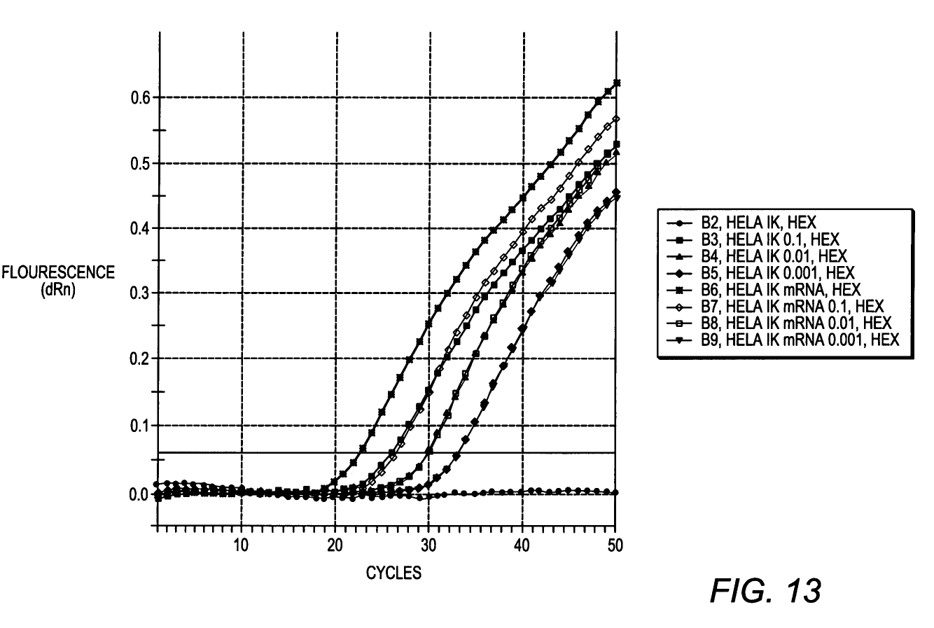

As can be seen from FIG. 13, detection of target mRNA sequences in purified mRNA and mRNA in cell lysates is essentially identical (with the exception of one cell lysate reaction, which failed). That is, the sensitivity of amplification and detection is the same, regardless of whether mRNA is isolated prior to amplification and detection. Thus, the present invention provides a simplified, more rapid, and less expensive method for identifying target sequences than a method currently widely practiced in the art (i.e., lysis and purification prior to PCR detection).

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and Examples be considered as exemplary only, with a true scope and spirit of the invention being indicated by the following claims.

1. A composition comprising Tris(2-carboxyethyl)phosphine (TCEP), at least one non-ionic detergent, and at least one nucleic acid.

2. The composition of claim 1, wherein the composition comprises at least one cell.

3. The composition of claim 1, wherein the composition comprises cell lysate.

4. The composition of claim 1, wherein the composition comprises 1-10 mM TCEP and 0.5%-10% of at least one non-ionic detergent.

5. The composition of claim 1, wherein the composition comprises 5 mM TCEP and 1% of at least one non-ionic detergent.

6. The composition of claim 1, wherein the non-ionic detergent is Triton X-100.

7. The composition of claim 1, wherein the pH of the composition is acidic.

8. A method of lysing a cell, said method comprising exposing the cell to a composition comprising TCEP and at least one non-ionic detergent for a sufficient amount of time for lysis to occur.

9. The method of claim 8, further comprising mechanically shearing the cells.

10. The method of claim 8, wherein the non-ionic detergent is Triton X-100.

11. The method of claim 8, further comprising storing the cell lysate composition.

12. A method of preparing a stabilized composition comprising at least one nucleic acid, said method comprising exposing at least one nucleic acid to a composition comprising TCEP and at least one non-ionic detergent.

13. The method of claim 12, further comprising maintaining the nucleic acid in the presence of the TCEP and non-ionic detergent for at least 4 hours.

14. A method of amplifying a nucleic acid of interest, said method comprising exposing the nucleic acid of interest to a composition comprising TCEP and at least one non-ionic detergent to make a mixture, and subjecting the mixture to conditions that result in amplification of the nucleic acid of interest.

15. The method of claim 15, wherein subjecting comprises performing a PCR amplification of the nucleic acid of interest.

16. The method of claim 15, further comprising, prior to amplifying, obtaining the nucleic acid of interest by lysing cells containing the nucleic acid of interest with the composition comprising TCEP and at least one non-ionic detergent.

17. A kit comprising, in packaged combination, a container containing a composition comprising TCEP and at least one non-ionic detergent, and a container containing at least one nucleic acid.

18. The kit of claim 17, wherein the nucleic acid comprises at least one primer for amplification of a nucleic acid sequence of interest.

19. The kit of claim 17, further comprising one or more containers containing some or all of the reagents necessary for amplification of a nucleic acid sequence of interest.

20. The kit of claim 17, further comprising one or more containers containing some or all of the reagents necessary for performing a PCR technique.
